(19) United States
(12) Patent Application Publication
(10) Pub. No.: US 2018/0299358 A1
(43) Pub. Date: Oct. 18, 2018

(54) CHEWING GUMS AND METHODS FOR EXTRACTING CHEMICALS FROM ORAL CAVITIES AND BREATH

(71) Applicant: VOLATILE ANALYSIS CORPORATION, Grant, AL (US)

(72) Inventors: Russell A. Bazemore, Grant, AL (US); Christopher P. Christensen, Sequin, TX (US); Mitchell R. White, Round Rock, TX (US)

(21) Appl. No.: 15/953,370

(22) Filed: Apr. 13, 2018

Related U.S. Application Data
(60) Provisional application No. 62/484,943, filed on Apr. 13, 2017.

(51) Int. Cl. G01N 1/40; G01N 1/22; A61B 10/00

(52) U.S. Cl. CPC G01N 1/405; G01N 1/22; A61B 2010/0087; G01N 2001/2244; A61B 10/0051

(57) ABSTRACT

A chewing gum has absorbent material, such as polydimethylsiloxane (PDMS), for absorbing or adsorbing volatile and non-volatile chemicals from an oral cavity or breath of a user. While the gum is being chewed, chemicals in the breaths and saliva of the user absorb or adsorb into the absorbent material. The absorbent material is then analyzed to determine the amount of chemicals absorbed or adsorbed into the absorbent material over time. Based on such analysis, various diseases or conditions can be detected or diagnosed.

### CROSS REFERENCE TO RELATED APPLICATION

This application claims priority to U.S. Provisional Application No. 62/484,943, entitled “Chewing Gum for Extracting Volatile Compounds” and filed on Apr. 13, 2017, which is incorporated herein by reference.

### RELATED ART

Advances in instrumentation have resulted in new generations of reliable, accurate, and precise tools for the scientist (analytical chemist, food scientist, biochemist, biologist). Technical advances have opened new areas for research including the field of metabolomics, the study of metabolites produced in the body related to disease. Metabolomics is a rapidly growing research field and promises to make disease detection and diagnosis less invasive and much more rapid. Difficult and time consuming procedures that currently require blood, stool, urine, or even more invasive tissue collection samples will be required much less frequently, or not at all. Sources of metabolites include blood, urine, feces, sweat, and breath. Breath analysis is challenging because compounds present are smaller (lower molecular weight, typically less than 300 g/mole), volatile (exist preferentially in the gas state), and reactive. Trace levels of metabolites in breath add another dimension of difficulty because of the quantity of breath needed to pull out sufficient mass of compound to permit detection is relatively large.

Current methods for breath collection for subsequent analysis include exhaling one or two breaths directly into an instrument, or collecting from 2 or 3 breaths to many (0.6 to 250 L) into a Tedlar bag—an air-tight bag made of Teflon, plastic, or other inert material. Problems with these methods, however, limit their usefulness. For example, very low levels, e.g., less than 1 part per trillion, of metabolites present in the amount of breath analyzed are not typically sufficient to detect, or are detected with difficulty, by the most sensitive instrumentation. Also, methods for breathing directly into an instrument are cumbersome, inconvenient, and require that the user and instrument be present in the same location.

For example researchers at Menssana Research have developed Breathscanner 2.5, an instrument that incorporates gas chromatography and a detector to identify volatiles from the breath of users (e.g., patients) who breathe directly into an interface with the instrument. This device is cumbersome, and results are dependent upon the amount of a substance present in breath collected in the short time a user exhales into the device. U.S. Pat. No. 5,465,728 describes a hand held device measuring breath components. While portable, this device appears to lack trace level detection capability. Other methods for measuring breath include US Patent Pub. No. 2008/0008666 A1, which describes a method for monitoring the effectiveness of oral malodor treatment by measuring for specific chemicals listed. It does not appear to allow for novel extraction and detection means. Finally, a major flaw associated with collecting large quantities of breath with a Tedlar bag is the problem of transferring the metabolite present in a large volume of air into an instrument while eliminating the dilution effect. This method provides no means for concentrating metabolites. Also with bags, some volatile metabolites are absorbed into the bag construction materials, or stick (adsorb) to the sides thus unavailable for detection and measurement.

Accordingly, a device is needed to extract low levels (trace levels) of volatile, semi-volatile, and non-volatile compounds from breath, for the purpose of advancing the field of breath metabolomics. Such a device would also be useful in dental, food and flavor sciences. For example, in dental science, oral health may be assessed by sampling metabolites present in the saliva and breath. Common maladies such as gum health may be diagnosed based on the presence and concentration of known metabolites generated by infection including compounds associated with foul odor such as carbon disulfide, methyl mercaptan, and dimethyl sulfide. In food and flavor sciences, flavor and taste are known as chemosenses, meaning the sense of taste and smell (flavors, tastes and fragrances) are the brain's interpretation of signals generated by interactions of chemicals (from foods and fragrances) with receptors in the mouth and nose. By detecting and measuring these chemicals in the oral cavity, improvements in flavor and fragrance technologies, duration, and efficacy may be successfully measured at a level currently unavailable. For example how long a product freshens your breath, or how long a product provides a pleasant taste may be more accurately assessed by measuring the time a breath freshening chemical resides in the oral cavity before being rinsed away in saliva or exhaled in air.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 illustrates an exemplary chewing gum for extracting chemicals from an oral cavity and breath in accordance with an exemplary embodiment of the present disclosure.

FIG. 2 illustrates a top view of the chewing gum depicted by FIG. 1.

FIG. 3 illustrates a side view of the chewing gum depicted by FIG. 1.

### DETAILED DESCRIPTION

The present disclosure generally pertains to chewing gums and methods for extracting volatile, semi-volatile, and non-volatile chemicals from an oral cavity and breath. A chewing gum for extracting chemicals in one exemplary embodiment is portable, convenient, and designed to easily fit in the oral cavity. It is composed of absorbent material that is intended to remain in the oral cavity for a period ranging from minutes to hours, although other time periods are possible. With each exhalation, air flows over the absorbents, and quantities of chemicals present in the breath are absorbed and/or adsorbed. As many (e.g., thousands) of breaths flow over the absorbent material, chemicals present in the breath and saliva are retained. Even trace levels of volatile chemicals present in the breath and saliva are sufficiently concentrated in the absorbent material over time to enable detection of these small amounts of chemicals via known analytical techniques. After a period of time, the chewing gum is removed from the oral cavity, and the absorbent material is analyzed by appropriate analytical instrumentation to determine the chemical compounds absorbed from the breath and oral cavity (e.g., saliva).

Various types of materials may be used to absorb and/or adsorb chemicals from the breath and oral cavity. Such materials include, but are not limited to, polydimethylsiloxane (PDMS), polyvinyl acetate, polyisoprene, styrene-butadiene rubber (SBR), polybutylene, polyacrylate, as well as other polymers that are known in the art, or may become known in the art, that are safe for use in oral cavities. Such materials may be used in different ratios in combination with one another or alone by themselves. Softening agents such as microcrystalline wax may also be utilized to provide a softer, easy to mold polymer. Additional absorbent materials that may be incorporated into the gum described herein include all forms of activated carbon with engineered pore sizes such as CarboPack or Carboxen materials, structures known as zeolites, an absorbent material called Tenax, and cyclodextrins.

Note that PDMS alone may be used as the absorbent material. One characteristic of PDMS is that it is hydrophobic. It does not bind water appreciably, but it does extract other volatile components present in a sample matrix (immersed in a liquid or from headspace) by absorption into the polymer liquid phase, making it ideal for use in the oral cavity environment.

Moreover, regardless of which type of absorbent material is used, the absorbent extracts and retains volatile, semi-volatile, and non-volatile components from the breath and oral cavity by absorption and adsorption. Forces and mechanisms responsible for the absorption and/or adsorption include Van der Waals forces, polarity, and hydrophobicity or hydrophillicity.

After absorption and/or adsorption, chemical components may be desorbed, analyzed, and measured by any of various types of analytical procedures and instruments. Such methods include, but are not limited to, thermal desorption and chemical desorption by exposure to solvent as with high performance liquid chromatography (HPLC). For thermal desorption, the absorbent is placed in a thermal desorption unit or heated chamber, equipped with inert gas flushing and temperature control. Upon heating the chamber, volatiles desorb from the absorbent material, are swept by inert gas (e.g., helium, nitrogen, argon) into a trap mechanism (e.g., a liquid nitrogen cooled cryo-trap, an absorbent material, or a combination thereof). The trap mechanism may be rapidly heated to release components and deposit them as a tight band on a capillary column for separation by a gas chromatograph (GC) and detection and measurement by a detector (e.g., mass spectrometer (MS), flame ionization, or flame photometric). Alternatively, the volatiles may be desorbed by solvent and analyzed by GC as previously described, or by HPLC. HPLC may utilize various detectors, such as MS, infra-red, ultraviolet, diode array, and/or other wavelength of electromagnetic radiation.

Data from the analysis may be used in a variety of ways. As an example, it may be determined that the presence of certain chemicals in certain quantities and/or a pattern of certain chemicals over time within the oral cavity and/or breath may indicate the presence of a certain disease or condition. Thus, the data may be analyzed to predict or diagnose whether a user, which could be a human or an animal, has or will have a certain disease or condition. By keeping the absorbent in the oral cavity for an extended period (e.g., several minutes or hours), even trace levels of chemicals can be concentrated in the absorbent allowing detection of such trace levels by conventional analytical equipment.

In one application, the absorbed chemicals are analyzed to determine whether the user has been exposed to (e.g., inhaled or otherwise consumed) certain chemicals. As a mere example, samples from a soldier may be analyzed to determine whether the soldier has been exposed to chemical weapons and, if so, to identify the type of chemicals to which he or she has been exposed. Various other types of conditions may be detected in other applications.

Exemplary embodiments of apparatuses and methods that may be used to extract chemicals from the oral cavity and breath are described by U.S. Pat. No. 9,480,461, issued on Nov. 1, 2016 and entitled “Methods for Extracting Chemicals from Nasal Cavities and Breath,” which is incorporated herein by reference. U.S. Pat. No. 9,480,461 describes an embodiment in which a chewing gum is used to extract chemicals from a user's oral cavity and breath. In this regard, the gum is composed of absorbent material and is chewable so that a user can place the gum into his or her oral cavity and chew the gum. During chewing, the gum is deformed, and saliva flow is stimulated. Chemicals present in saliva, oral cavity, and breath are extracted and absorbed or adsorbed into the absorbent material. After chewing for a desired time period sufficient to extract chemicals from the breath and saliva, the gum is then removed from the oral cavity and analyzed. If desired, the gum may be cut or otherwise re-shaped or arranged in an effort to facilitate analysis.

The chewing gum may be composed of different combinations of heat resistant polymers, including polydimethylsiloxane (PDMS), polyvinyl acetate, polyisoprene, styrene-butadiene rubber (SBR), and polybutylene. Additionally a microcrystalline wax may be utilized as a softener. Various known materials typically used in conventional chewing gums may be used to manufacture the gum. Further, like other conventional gum products, the chewing gum can have many different sizes and shapes, and it can be manufactured using other known techniques for manufacturing conventional chewing gum.

PDMS may be incorporated into the chewing gum by placing PDMS and other polymers or other types of material into a mixer capable of providing sufficient shearing force such as a heated Z-blade mixer. In other embodiments, other types of absorbent material can be used. Contents are blended for a period (e.g., about 15 to 30 minutes) to provide a homogenous product and heated to temperatures that range from around 50 degrees Celsius (C.) to around 200 degrees C. Mixing is conducted until the formulations result in a gum that is sufficiently malleable that it may be chewed by most healthy individuals. Edible wax may be added to increase gum softness. Hydrophobic and hydrophilic nature of gum can be adjusted by selection and concentration of co-polymer utilized. Other ingredients which may be added to the gum-like product of the present disclosure include other absorbents such as activated carbon, Carbopack, carboxen, edible wax for softening.

In some embodiments, a chewing gum may be formed by adding PDMS or other absorbent material to conventional chewing gum. However, chewing gum is typically a complex material that includes many ingredients, including several different polymers, to provide a desired texture and taste to the user. Many of these ingredients introduce impurities that interfere with testing. In this regard, when heated, the impurities may desorb from the gum and then mask chemicals from the user's oral cavity and breath making it particularly difficult to detect trace levels of certain chemicals. Further, during testing using certain techniques, such as gas chromatography or liquid chromatography, the material is typically heated to a high temperature, such as around 200 to 300 degrees Celsius, and many of the polymers and other ingredients in conventional gum degrade at such high temperature. What is needed, therefore, is a chewing gum for absorbing or adsorbing even trace levels of chemicals from a user's oral cavity and breath without introducing significant impurities yet capable of withstanding high temperatures, such as between about 200 and 300 degrees Celsius.

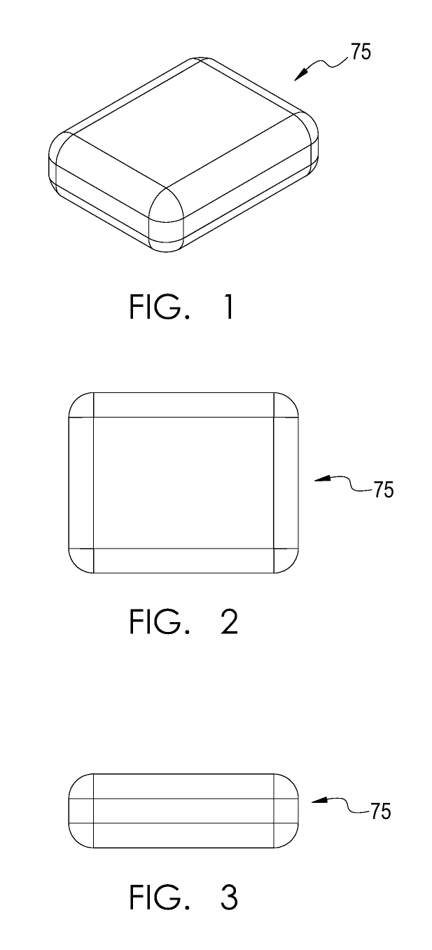

FIGS. 1-3 depict a chemical extraction gum 75 that is chewable, like conventional chewing gum. The chewing gum 75 is composed of absorbent material, such as PDMS or other material for absorbing and/or adsorbing chemicals from the oral cavity and breath, and has a texture conducive to chewing over an extended period, such as minutes or hours. While the gum 75 is being chewed, it absorbs and/or adsorbs chemicals from the user's breath and oral cavity (e.g., saliva). After exposure for a desired duration, such as several minutes or hours, the gum 75 is removed from the oral cavity and then analyzed to determine the chemicals and the concentrations of the chemicals absorbed and/or adsorbed by the gum 75. The data from such analysis may then be used for a variety of purposes, such as diagnosing a disease or condition of the user (e.g., patient) or identifying a marker or predictor of a disease or condition.

In some embodiments, the gum 75 is composed of a combination or mixture of an absorbent material, such as PDMS, for absorbing or adsorbing chemicals and another material, such as clay, that when combined with the absorbent material imparts a texture similar to conventional chewing gum. PDSM and clay are desirable for use in the gum 75 for a variety of reasons. In this regard, both PDMS and clay are stable at high temperatures above 200 degrees Celsius. Further, clay has a relatively high surface area and aspect ratio that modifies the rheology of the gum 75 to impart a texture similar to conventional chewing gum without having to use polymers or other materials that might otherwise introduce impurities or degrade at high temperature. Moreover, the texture of the gum 75 can be controlled through selection of the viscosity of the absorbent material, such as PDMS, the aspect ratio of clay, and the amount of clay added to the absorbent material. In some embodiments, the viscosity may be between about 20,000 centistokes (cSt) and 100,000,000 cSt. Moreover, clay may be mixed with or otherwise added to the PDMS material until a desired texture for the gum 75 is reached.

PDMS is very good absorber of non-polar molecules but is not quite as effective at absorbing polar molecules. In some embodiments, additional absorbent materials for absorbing or adsorbing chemicals may be introduced. Examples of such additional absorbent materials may include materials that are good at absorbing or adsorbing polar molecules, such as activated carbon (including engineered activated carbon particles with specific pore sizes), zeolites, Tenax, beta cyclodextrins, and carbon nanoparticles.

In some embodiments, montmorillonite is used as the clay material that is combined with the absorbent material to form the gum 75. However, other types of clay materials are possible in other embodiments. As an example, the clay material may include kaolinite, montmorillonite, smectite, illite, chlorite or any combination thereof. The concentration of clay material may be from less than 1% to 75% or more.

If desired, gum flavors may be added that do not unacceptably interfere with analytical measurement of chemicals. Flavors may include less volatile substances, such as those that impart sweet, sour, spicy (capsaicin), salty, bitter, astringent, etc. To help avoid interference with testing, it may be desirable to use flavors that quickly chew out, or come out of the gum 75, and are dissolved in saliva and swallowed. Use of an added flavor component may also serve to function as an analytical internal standard for quantitative measurements of various compounds.

In addition, the gum 75 may contain chemical compounds or functionality that derivatizes, or slightly modifies the chemical structure or bonding of/to target biomarkers or hard-to-capture metabolites important in disease and improves analytical detection and measurement. The gum 75 may contain chemicals that, on interaction with specific compounds present in the oral cavity, enhance the detection of the target disease.

The gum 75 may also have additives that affect the pH level of the gum 75 or the oral cavity as may be desired. As an example, the gum 75 may adjust/buffer the pH of the oral cavity when chewed to enhance metabolite collection, and/or reduce interferences. The gum 75 may enhance the collection of a class of chemicals known as “amines” by modifying pH to acidic levels and use basic pH to release amines after capture. The gum 75 also may enhance the collection of a class of chemicals known a “volatile acids” by modifying pH to basic levels and use acidic pH to release acids after capture. Oral cavity pH may also be adjusted by use of an acidic or basic rinse to enhance collection of amines or volatile acids. Some additives, such as citric acid, may be added to the gum 75 to modify the pH of the gum 75 while also providing a flavor that stimulates saliva production in the oral cavity. Such stimulation of saliva production may greater absorption or adsorption of trace levels of chemicals into the gum 75.

At high temperature, PDMS material may release relatively high amounts of impurities, such as siloxane, that may unacceptably interfere with the detection of trace amounts of certain chemicals. In some embodiments, the PDMS is purified prior to mixing or otherwise combining with the clay material in order to remove various impurities, including siloxane. An exemplary process for purifying the PDMS in order to remove significant amounts of siloxane and other impurities from the PDMS is described in more detail below.

The PDMS is fully dissolved in dichloromethane or DCM (also known as methylene chloride). A ratio of approximately 1:5 weight/weight (PDMS:DCM) may be used, but other ratios are possible in other embodiments. The solution is mixed thoroughly and methanol is added to the PDMS:DCM solution. Approximately twice as much methanol relative to the original weight of the PDMS:DCM solution may be added, but other amounts of methanol may be added in other embodiments. PDMS precipitates from the solution as a gel, and a percentage of impurities remain in the solution. Excess DCM and methanol are poured off and removed leaving only the PDMS gel precipitate. The PDMS gel is baked in an ultra-high purity (UHP) nitrogen atmosphere at temperatures exceeding 200 degrees Celsius for more than 2 hours to remove residual solvent and additional PDMS impurities. A vacuum oven may be used. The aforementioned process may be repeated if desired to help further remove impurities remaining in the PDMS. Note that use of HPLC grade solvent purity is desired. In addition, various modifications and changes to the purification process would be apparent to a person of ordinary skill upon reading this disclosure.

By selecting materials and purifying the absorbent material according to the techniques described herein, the resulting chewing gum may have various properties that make it desirable and conducive for use in extracting chemicals from the oral cavity and breath. In this regard, the gum 75 may be chewed and remain in the oral cavity absorbing or adsorbing chemicals for an extended time such as many minutes or hours, thereby facilitating detection of trace levels of certain chemicals. The gum 75 is substantially clean in that it introduces very low amounts of impurities that would otherwise be released at high temperature and interfere with testing. In addition, the gum 75 exhibits the general texture (viscosity, rheology) of conventional chewing gum and will remain elastic and intact (will not significantly decompose, fragment into small pieces, dissolve, or lose majority of bulk mass in the user's mouth) while chewing for extended periods of time, such as up to at least about 12 hours. The gum 75 may also remain stable at high temperatures, such at 200 degrees Celsius or more. As an example, the gum 75 will not thermally degrade at a temperature of about 200 degrees for at least 30 minutes or longer, thereby making the gum 75 suitable for use with thermal desorption techniques utilized in analytical instrumentation, such as gas chromatography and liquid chromatography.

Now, therefore, the following is claimed:

1. A chewing gum for extracting chemicals from an oral cavity or breath of a user, comprising:
polydimethylsiloxane; and
clay.
